(19) United States
(12) Patent Application Publication
(10) Pub. No.: US 2023/0384308 A1
(43) Pub. Date: Nov. 30, 2023

(54) HIGH-THROUGHPUT FORCE-DEPENDENT CELLULAR RESPONSE ASSAY USING SPECTRALLY ENCODED SMART BEADS

(71) Applicants: CZ Biohub SF, LLC, San Francisco, CA (US); The Board of Trustees of the Leland Stanford Junior University, Stanford, CA (US)

(72) Inventors: Yinnian Feng, Menlo Park, CA (US); Adam White, Palo Alto, CA (US); Polly M. Fordyce, Menlo Park, CA (US); Xiang Zhao, Menlo Park, CA (US); K. Christopher Garcia, Menlo Park, CA (US)

(21) Appl. No.: 18/250,723

(22) PCT Filed: Oct. 29, 2021
(86) PCT No.: PCT/US2021/057263

§ 371 Date: Apr. 26, 2023

Related U.S. Application Data
(60) Provisional application No. 63/108,162, filed on Oct. 30, 2020.

(51) Int. Cl. G01N 33/569; G01N 33/543; G01N 33/542

(52) U.S. Cl. CPC G01N 33/56977; G01N 33/54353; G01N 33/542

(57) ABSTRACT

Described herein are thermo-responsive microbead compositions, methods of making such microbead compositions, and method employing the microbeads to evaluate cellular responses elicited by mechanical force.

### CROSS-REFERENCE TO RELATED APPLICATIONS

This application claims benefit of U.S. provisional application No. 63/108,162, filed Oct. 30, 2020, which is herein incorporated by reference for all purposes.

### STATEMENT AS TO RIGHTS TO INVENTIONS MADE UNDER FEDERALLY SPONSORED RESEARCH AND DEVELOPMENT

This invention was made with Government support under contract GM123641 awarded by the National Institutes of Health. The Government has certain rights in the invention.

### BRIEF SUMMARY OF THE INVENTION

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the illustrative embodiments of the present invention are discussed below.

In one aspect, provided here is a method of identifying a binding moiety that binds to a target protein on the surface of a cell and mediates a force-induced signal, the method comprising:
(i) generating an arrayed configuration comprising a plurality of analysis compartments, wherein each analysis compartment in at least a portion of the arrayed configuration comprises (a) single cell and (b) a thermo-responsive microbead comprising a thermo-responsive polymer, said microbead having a lanthanide spectral signature and, immobilized to the microbead, a binding moiety that targets a mechanosensitive molecule on the surface of the single cell;
wherein the microbead in an analysis compartment has a lanthanide spectral signature that differs from lanthanide spectral signatures of microbeads in other analysis compartments of the arrayed configuration, and the binding moiety immobilized to the microbead differs in structure or in density compared to binding moieties attached to microbeads in other analysis compartments;
(ii) incubating microbeads in the analysis compartments at a temperature of between about 25° C. and about 40° C.; (iii) modulating the temperature to cross the phase transition temperature of the thermo-responsive polymer such that the diameter of the microbead is increased by about 1% to about 5%, thereby applying mechanical force to the cell; (iv) measuring a signal generated in individual cells in the analysis compartments; (v) determining the lanthanide spectral signature of a microbead contained in an analysis compartment in which a signal is detected; and (vi) identifying the binding moiety associated with the microbead present in the analysis compartment in which a signal is measured. In some embodiments, modulating the temperature comprises cooling the microbeads from a temperature above the phase-transition temperature of the thermos-responsive polymer to a temperature below the phase transition temperature of the thermo-responsive polymer, wherein the thermo-responsive polymer increases in size when cooled from a temperature above the phase-transition temperature to a temperature below the phase transition temperature. In some embodiments, the thermo-responsive polymer comprises poly(N-isopropylacrylamide). In some embodiments, e.g., where the thermo-responsive microbead comprises the thermos-responsive polymer poly(N-isopropylacrylamide), step (ii) comprises incubating the microbeads at a temperature in a range from about 37° C. to about 40° C. and/or step (iii) comprises decreasing the temperature to a temperature of about 32° C. to about 34° C. In some embodiments, step (ii) comprises incubating the microbeads at about 37° C. and step (iii) comprises decreasing the temperature to about 34° C. In other embodiments, modulating the temperature comprises heating the microbeads from a temperature below the phase-transition temperature of the thermos-responsive polymer to a temperature above the phase transition temperature of the thermos-responsive polymer, wherein the thermo-responsive polymer increases in size when heated from a temperature below the phase-transition temperature to a temperature above the phase-transition temperature. In some embodiments, the arrayed configuration comprises compartments in which the microbeads have different phase transition temperatures. In some embodiments, the mechanosensitive molecule comprises a T-cell receptor. Thus, for example, in some embodiments, the binding moiety immobilized on the thermo-responsive microbead is a peptide-loaded major histocompatibility complex (MHC) protein and the single cell in each compartment is a T cell, e.g., a CD8+ T cell or a CD4+ T cell. In some embodiments, the peptide loaded onto the MHC protein is a candidate T-cell epitope, e.g., from a cancer antigen. In some embodiments, step (iv) comprises measuring calcium flux. In some embodiments, the binding moiety is immobilized to the microbead via interaction of biotin and streptavidin.

In a further aspect, provided herein is a thermo-responsive microbead having a lanthanide spectral signature and a binding moiety immobilized to the said microbead, where the binding moiety targets a mechanosensitive molecule on the surface of a cell and wherein the microbead comprises a thermo-responsive polymer having a phase transition temperature in the range of about 25° C. to about 45° C. In some embodiments, the thermo-responsive microbead comprises the thermo-responsive polymer poly(N-isopropylacrylamide). In some embodiments, the binding moiety targets a T-cell receptor. Thus, for example, in some embodiments, the binding moiety immobilized on the thermo-responsive microbead is a peptide-loaded MHC protein, wherein the peptide comprises a candidate T cell epitope, e.g., a CD8+ T-cell epitope or a CD4+ T-cell epitope. In some embodiments, the candidate T-cell epitope is from a cancer antigen. In some embodiments, the binding moiety is immobilized to the microbead via interaction of biotin and streptavidin.

In an additional aspect, provided herein is an arrayed configuration comprising a plurality of analysis compartments, wherein each analysis compartment in at least a portion of the arrayed configuration comprises (a) a single cell and (b) a thermo-responsive microbead, e.g., as described in the preceding paragraph, wherein the lanthanide spectral signature of a microbead differs from the lanthanide spectral signature of microbeads in other analysis compartments, and the binding moiety immobilized to the microbead differs in structure or density from the binding moieties immobilized to microbeads in other compartments; and/or the arrayed configuration comprises analysis compartments in which microbeads present in different compartments differ in phase transition temperature.

In another aspect, provided herein is a method of identifying an major histocompatibility complex epitope that activates a T-cell, the method comprising:
i) generating an arrayed configuration comprising a plurality of analysis compartments, wherein at least a portion of the arrayed configuration comprises (a) a single T-cell and (b) a thermo-responsive microbead comprising a thermo-responsive polymer and a peptide-loaded MHC complex immobilized to the microbead,
wherein a microbead in an analysis compartment has a lanthanide spectral signature that differs from the lanthanide spectral signature of microbeads in other analysis compartments, and the peptide-loaded MHC complex comprises a peptide that differs in sequence compared to peptides of peptide-loaded MHC complexes in other compartment, and/or the peptide-loaded MHC complex is present on the microbead at a different density compared to peptide-loaded MHC complexes immobilized to single T-cells in other analysis compartments;
(ii) incubating microbeads of (i) at a temperature of between about 25° C. and about 40° C.;
(iii) modulating the temperature to cross the phase transition temperature of the thermos-responsive polymer such that the diameter of the thermos-responsive microbead is increased by about 1% to about 5%, thereby applying shear force to the cell;
(iv) measuring a signal generated by individual T-cells in the analysis compartments;
(v) determining the lanthanide spectral signature of a microbead contained in an analysis compartment in which a signal is detected; and
(vi) identifying the sequence of the peptide of the peptide-loaded MHC complex and/or the density of the peptide-loaded MHC complex associated with the microbead in an analysis compartment in which a signal is detected, thereby identifying a T-cell epitope that activates a T cell. In some embodiments, the thermo-responsive polymer comprises poly(N-isopropylacrylamide). In some embodiments, step (ii) comprises incubating the microbeads at a temperature of about 37° C. to about 40° C.; and/or step (iii) comprises decreasing the temperature to about 32° C. to about 34° C. In some embodiments, step (i) comprises incubating the microbeads at a temperature of about 37° C. and step (ii) comprises decreasing the temperature from about 37° C. to about 34° C. In some embodiments, step (iv) comprises measuring calcium flux. In some embodiments, the T-cell is a CD8+ T-cell or a CD4+ T-cell. In some embodiments, the arrayed configuration comprises at least a first compartment that contains a peptide-loaded MHC complex in which the peptide sequence is the same as the sequence of a peptide-loaded MHC complex present in a second compartment and the density of the peptide-loaded MHC complexes immobilized to the microbead in the first compartment is different from the density of the peptide-loaded MHC complexes immobilized to the microbead in the second compartment. In some embodiments, the arrayed configuration comprises at least a first compartment that contains a peptide-loaded MHC complex in which the peptide sequence and the density of the peptide-loaded MHC complex immobilized to the microbead is the same as the peptide sequence and the density of the peptide-loaded MHC complex in a second compartment, and the microbead in the first compartment differs in its phase transition temperature compared to the microbead in the second compartment.

### BRIEF DESCRIPTION OF THE DRAWINGS

FIG. 1A-1B: Overview of Biomechanically Assisted TCR Triggering for Large-scale Exogenous-pMHC Screening (BATTLES). (1A) BATTLES involves: 1) ‘smart beads’ that are spectrally encoded (with a 1:1 linkage between embedded spectral code and presented pMHC sequences, pMHC concentration, force, or these interlaced parameters) and made of a swellable polymer (to allow application of precise loads) and 2) a microwell array that pairs ‘smart’ beads and T cells for high-throughput monitoring of T cell activation after the application of load. (1B) Images at various magnifications showing microwell device (left), well array (middle), and smart beads and cells co-localized in microwells.

FIG. 2A-2G: Force calibration of ‘smart’ beads. (2A) Expansion force loaded on the T cell through the heating-cooling cycle can be well-calibrated. In the equation, R_{cell} is the radius of the T cell (4 μm on average), K refers to the modulus of rigidity of the ‘smart bead’, R_{bead} is the radius of the bead in the “cold” state and ΔR_{bead} is the radius change of the bead from 37° C. to 34° C. The ramping force velocity was obtained by dividing the force over 60 s. (2B) ‘Smart bead’ matrix was made from thermo-responsive pNIPAM. (2C) Parallel microfluidic chip for high-throughput production of spectrally encoded ‘smart’ beads (100,000 beads/min with UV polymerization off-chip). (2D) ‘Smart’ bead radius increases ˜4% upon cooling. (2E) Representative thermo-responsive curves of the ‘smart’ beads. Code 11 was used as an example. (2F) Representative radius changes of ‘smart’ beads at the end of each thermo-cycle. Code 11 was used as an example. (2G) Modulus of rigidity (K) of ‘smart’ slabs with same composition as beads measured by rheometer.

FIG. 3A-3D: Peptide identity associated with the spectrally encoded ‘smart’ bead. (3A) Spectral codes (No. 1 to 21) embedded within ‘smart’ beads are easily resolved (21 codes by varying the ratio of Dy and Sm relative to Eu). (3B) Force calibration per code. (3C) UV-exchange method for desired pMHC generation. (3D) Peptides used for TCR55 and TCR589 cell systems displayed on ‘smart’ bead surface with ˜3-4 pMHCs per cell.

FIG. 4A-4D: Activation responses of TCR55 system with 21 peptides. (4A) Cartoon schematic showing workflow and expected results. (4B) Representative images and traces demonstrating force- and sequence-dependent activation of T cells characterized by the fluorescence change (I/I₀) using expansion of antigenic pMHC-coated ‘smart beads’. (4C) Statistical resampling of the positive cells shows that VPLTEDALL was the most active among the tested (p-value<0.001). (4D) Reproducibility of the BATTLES assay in TCR55 system. Note that the “outlier” VPLTEDALL is far away from the cluster of other peptides.

FIG. 5A-5B: Activation responses of TCR589 system with 21 peptides. (5A) Statistical resampling of the positive cells shows IPLTEEAEL was the most active among all the tested peptides (p-value<0.001). (5B) Reproducibility of the BATTLES assay in TCR589 system. Note the “outlier” of IPLTEEAEL is far away from the cluster of other peptides.

FIG. 6A-6D: Force multiplex assay. (6A) Expansion force from the ‘smart’ bead can be tuned by changing the amount of hydrophilic monomer (sodium acrylate) component of the bead matrix. (6B) Representative thermo-responsive curves of the ‘smart’ beads with different amount of sodium acrylate added. (6C) Statistical resampling of the positive cells shows a ‘catch bond’ phenomena for VPLTEDALL and VPITEDSQL. (D) Reproducibility of the force multiplex BATTLES assay in TCR55 system. Note the “outliers” are VPLTEDALL (ALL) with optimal force (O_ALL) and low force (L_ALL), and VPITEDSQL (SQL) with low force (L_SQL). Labels in D are Low force (L), optimal force (O), high force (H), VPLTEDALL (ALL), VPLTEDAEL (pep), VPITEDSQL (SQL) and IPLTEEAEL (HIV).

FIG. 7A-7B: pMHC concentration-dependence assay. (7A) Statistical resampling of the positive cells shows a loss of specificity towards peptide sequences at higher pMHC concentrations (from 1×, 10× to 50×). (7B) Reproducibility of the pMHC concentration-dependent BATTLES assay in TCR55 system.

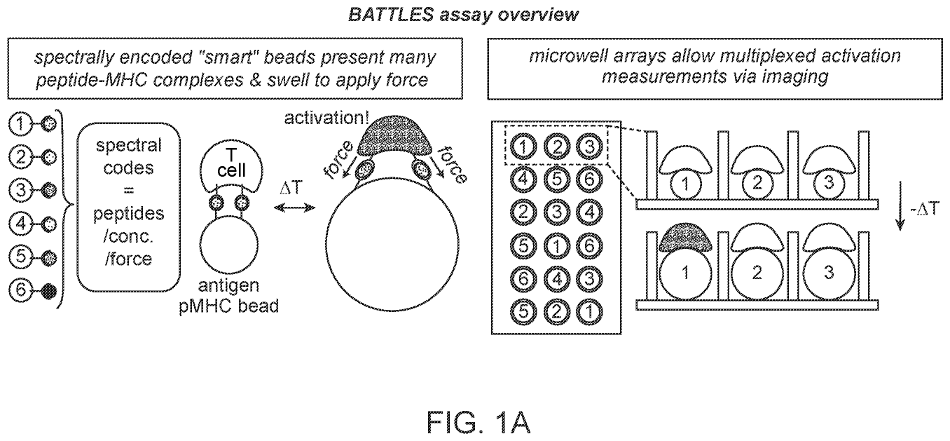

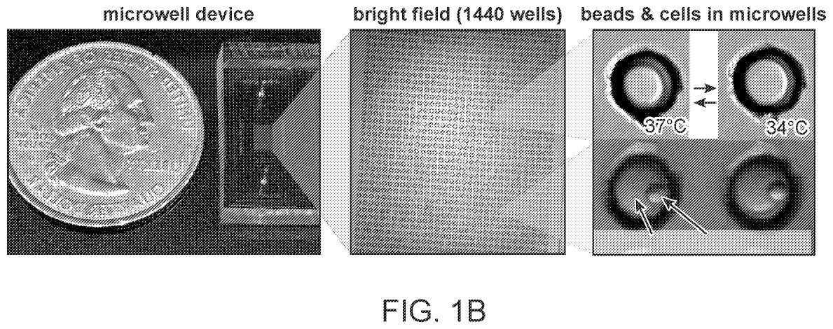

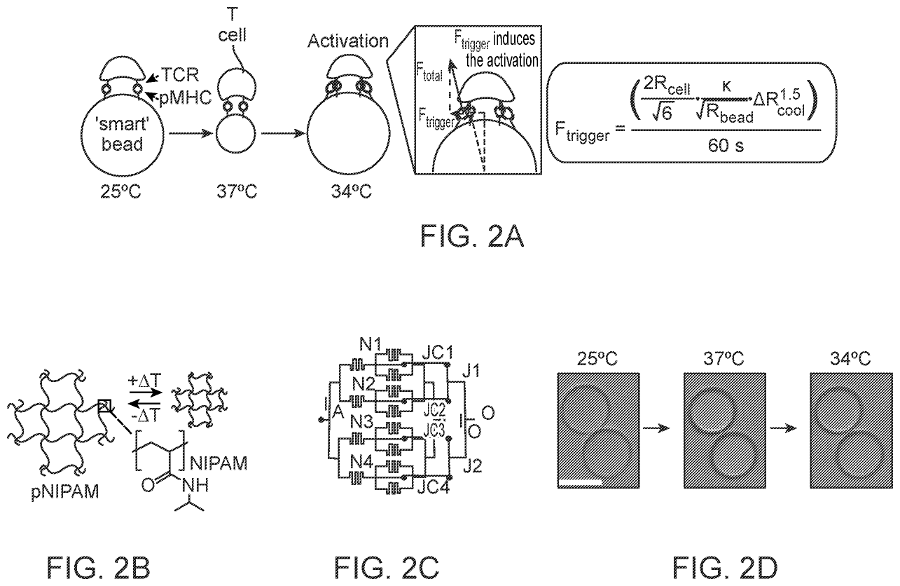

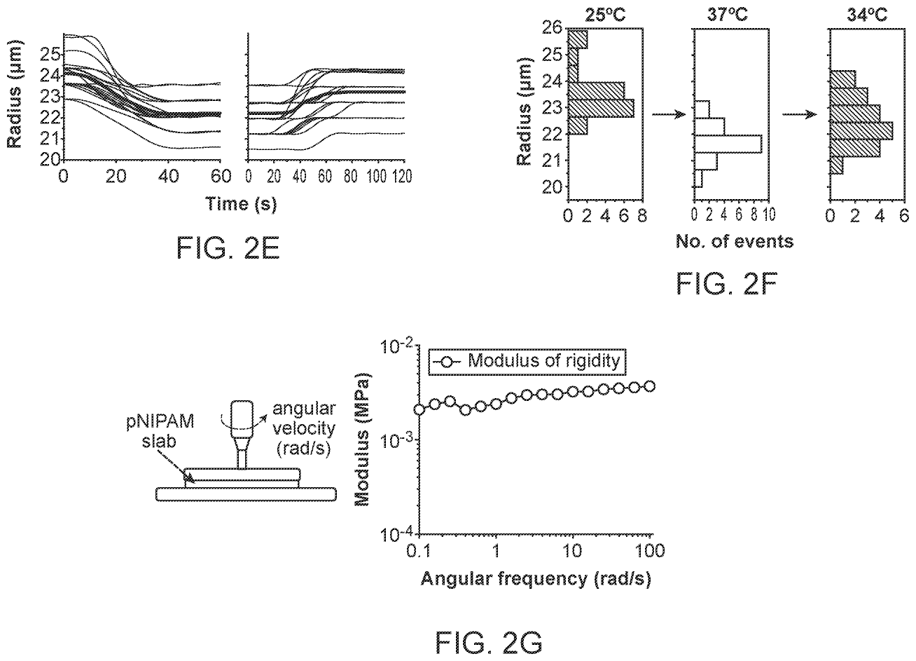

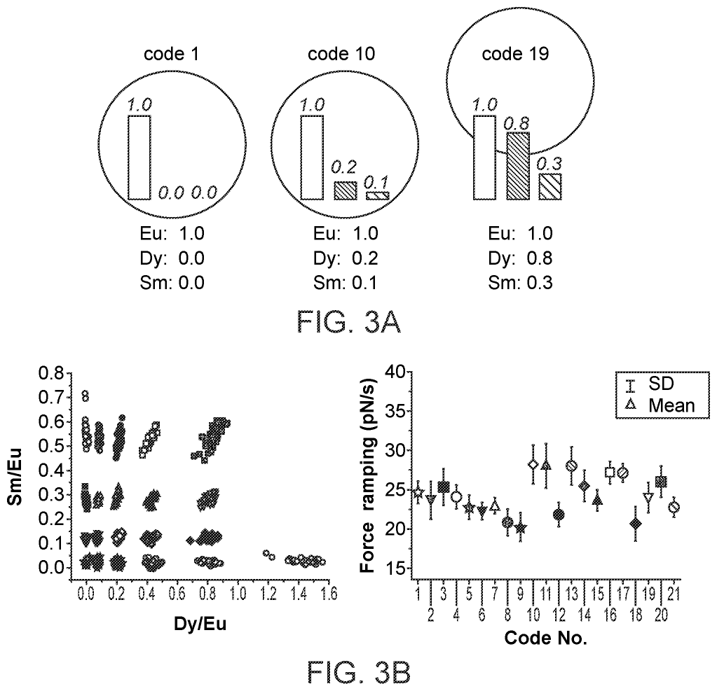

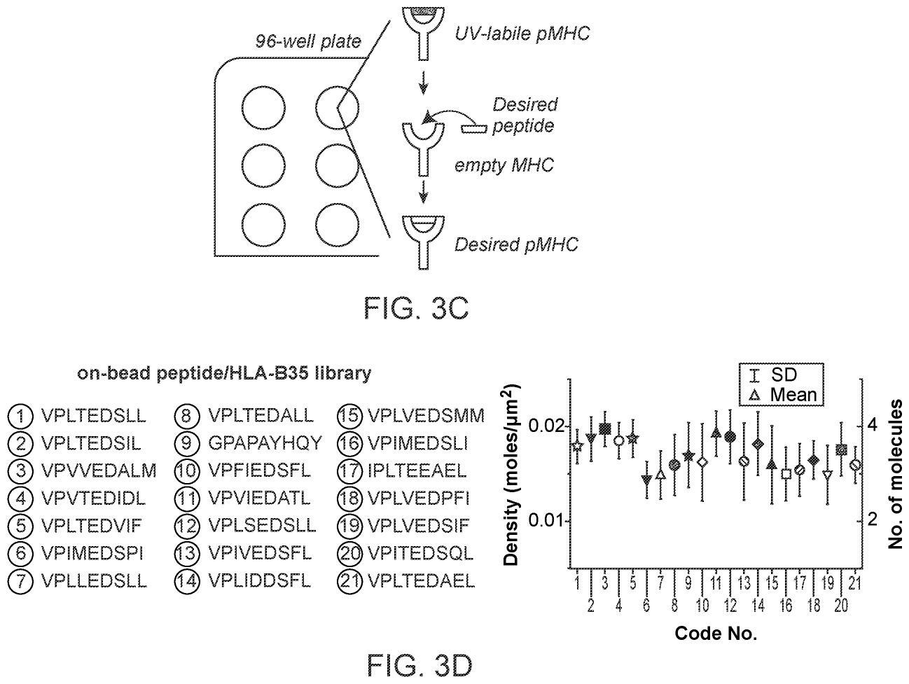

### DETAILED DESCRIPTION OF THE INVENTION

### Terminology

As used herein, the terms “a”, “an”, and “the” can refer to one or more unless specifically noted otherwise.

The terms “about” and “approximately” as used herein shall generally mean an acceptable degree of error for the quantity measured given the nature or precision of the measurements. For example, exemplary degrees of error for temperature may be less than 5%, e.g., 4%, 3%, 2%, 1%, or 0.5% of a given value or range of values. Any reference to “about X” or “approximately X” specifically indicates at least the values X, 0.95×, 0.96×, 0.97×, 0.98×, 0.99×, 1.01×, 1.02×, 1.03×, 1.04×, and 1.05×. Thus, expressions “about X” or “approximately X” are intended to teach and provide written support for a claim limitation of, for example, “0.98×.”. Numerical quantities given herein are approximate unless stated otherwise, meaning that the term “about” or “approximately” can be inferred when not expressly stated. When “about” is applied to the beginning of a numerical range, it applies to both ends of the range.

The term “microbead” and the related terms refer to a particle having one or more dimensions (such as length, width, diameter, or circumference) of about 1000 μm or less, but greater than about 1 m, greater than about 5 m, or great than about 10 μm. For example, in some embodiments, a particle may have one or more dimensions of about 900 μm or less, about 800 μm or less, about 700 μm or less, about 600 μm or less, about 500 μm or less, about 400 μm or less, or about 300 μm or less. In some embodiments, the microbead has one or more dimensions less than about 200 μm or less than 100 μm. In some embodiments, the microbead has one or more dimensions less than 90 μm or less, about 80 μm or less, about 70 μm or less, or about 60 μm or less, about 50 μm or less, about 40 μm or less, or about 30 μm or less, where the microbead has a minimum size of about 1 μm or about 10 μm. The microbead may have a generally spherical shape. Thus, in some embodiments, the dimension ranges specified are microbead diameter. In some embodiments, the microbead has a diameter from about 0.2 μm to about 1000 μm. In some embodiments, the microbead has a diameter from about 1 μm to about 500 μm. In some embodiments, the microbead has a diameter from about 5 μm to about 200 μm. In some embodiments, the microbead has a diameter from about 10 μm to about 100 μm. In some embodiments, the microbead has a diameter from about 30 μm to about 60 μm. In some embodiments, the microbead diameter is about 40 μm to about 50 μm.

The terms “plurality” or “population,” when used in connection with microbeads (for example, as in “a plurality of microbeads” or “a population of microbeads”), refer to groups of microbeads (that is, more than one microbead) including various numbers of microbeads. For example, a plurality or a population of microbeads may include 2 or more, 10 or more, 100 or more, 500 or more, 10³ or more, 10⁴ or more, 10⁵ or more, 10⁶ or more, or 10⁷, or more microbeads. In some embodiments, each microbead in a plurality or a population of microbeads may have approximately the same one or more dimensions. For example, individual microbeads may have a diameter such that the diameter variation as measured by coefficient of variance (CV), when compared at a specific temperature, of the members of the plurality or a population of microbeads is from 4% to 10%, e.g., 6 to 7%.

The term “lanthanide” refers to elements 57-71 of the periodic table, namely lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). The term “lanthanide” can also refer to combinations of lanthanide elements, compounds containing lanthanide elements or their combinations, or ions containing lanthanide elements or their combinations.

“Lanthanide encoded” or “spectrally encoded” microbeads described in the present disclosure contain lanthanide nanoparticles (Lns) and possess a detectable spectral signature, which is a combination of luminescent signals in the range of 350-850 nm emitted from lanthanide nanoparticles contained in a single microbead upon excitation with an appropriate wavelength of light, for example, UV light (such as 292 nm for excitation of down-converting lanthanides) or IR light (such as 980 nm for excitation of up-converting lanthanides). The luminescence intensity at a characteristic wavelength or wavelengths (for example, 620 nm, 630 nm, or 650 nm) for a particular lanthanide (for example, Eu) indicates the presence and quantity of the particular lanthanide in the source (for example, a microbead) from which the spectral signature originates. A “lanthanide encoded” or “spectrally encoded” microbead may include one or more different types of lanthanide nanoparticles, for example, 2 or more, 3 or more, 4 or more, 5 or more, 6 or more, 7 or more, 8 or more, 9 or more, or 10 or more, wherein each lanthanide nanoparticle has a different luminescence emission spectrum upon excitation. Signals from the combined luminescence spectra make up the spectral signature of a particular microbead, and are mapped to a unique spectral signature code (or “spectral code”) during code deconvolution. Lanthanide nanoparticle spectra are typically characterized by narrow emission bands (also referred to as “signals”) in the visible region, making one species of material easily distinguishable from another. A lanthanide spectral signature of a microbead can therefore be designed based on the particular identity and relative amounts of lanthanides in the microbead. Lanthanide spectral signatures of microbeads described in the present disclosure can include one or more of an Eu signal, a Dy signal, an Sm signal, a Ce signal, a Tb signal, a La signal, a Pr signal, an Nd signal, a Gd signal, an Ho signal, an Er signal, a Tm signal, a Yb signal, a Pm signal, and a Lu signal.

The term “amino acid” encompasses naturally occurring amino acids as well as non-naturally occurring amino acids, including amino acid analogs and derivatives. Amino acids include naturally occurring proteogenic L-amino acids; D-amino acids; chemically modified amino acids such as amino acid analogs and derivatives; naturally occurring nonproteinogenic amino acids such as norleucine, p-alanine, or ornithine; and chemically synthesized compounds having amino acid characteristics.

The terms “peptide,” “polypeptide” or “protein” are used herein interchangeably to refer polymer of amino acids linked by native amide bonds and/or non-native amide bonds. Peptides, polypeptides or proteins may include moieties other than amino acids (for example, lipids or sugars). Peptides, polypeptides or proteins may be produced synthetically or by recombinant technology.

### Overview of the Disclosure

In one aspect, the present disclosure provides microbeads containing thermo-responsive polymers that undergo rapid, reversible changes in size with changes in temperature. Shrinking and/or expansion of the bead matrix resulting from temperature change can be used to apply mechanical force to a cell that is contacted with the bead. Thus, for example, a thermo-responsive polymer, poly(N-isopropylacrylamide) (NIPAM), can be used as a bead matrix to evaluate force-mediated cellular responses.

Cells that respond to external forces via targeted mechanical stimulation applied to specific receptors (receptor-mediated mechanosensing) may be evaluated using the methods of the present disclosure. For example, adhesion molecules such as cadherin and integrins are components of mechanosensing machineries that mediate cellular response to the changing mechanical environment. In some embodiments, cells can internally generate and exert forces on cell surface receptors. For example, changes in cytoskeletal components can be transmitted to T- or B-cell receptors bound to peptide-major histocompatibility complex (pMHC) molecules or antibodies anchored to a surface during signaling activation. Accordingly, in some embodiments T cells or B cell responses to mechanical force are evaluated using methods as described herein.

In some embodiments, force-mediated responses are evaluated in cells that respond to mechanical transduction through a mechanism other than binding of an agent to a receptor on the surface of a cell. For example, cells having mechanically gated ion channels can respond to mechanical forces such as sound waves that exert mechanical loads on hair bundles, skin cells, muscle cells, and other deformation signals to generate a cellular response. Accordingly, thermo-responsive microbeads of the present disclosure can be used to evaluate mechanical forces applied to such cells.

In some embodiments, the thermo-responsive microbeads comprise lanthanide particles, thus allowing for determination of single cells responses to mechanical force when employed in an arrayed configuration.

Thus, in some aspects, the present disclosure provides methods of preparing thermo-responsive microbeads, compositions comprising such microbeads, and methods of using thermos-responsive microbeads for the evaluation of force-mediated cellular responses.

### Preparation of Microbeads

### Thermo-Responsive Polymers

Microbeads employed for the measure of force-mediated cellular responses of present disclosure comprise a hydrogel generated using a water soluble polymeric material that undergoes rapid, reversible size changes due to a hydrophilic/hydrophobic phase transition with temperature changes that cross a critical solution temperature (also referred to in the disclosure as “a phase transition temperature”) for the polymer. Such polymers are referred to herein as “thermo-responsive polymers”. A bead that changes in diameter upon changing the temperature at which the bead is incubated applies a load, i.e., force, to a cell that is incubated with the bead during or after the temperature change. Use of co-polymeric hydrogels allows tuning of transition temperature by varying the amount of the polymer ingredients. Polymeric microbeads comprising thermo-responsive polymers are generally prepared using polymers that will provide a change in diameter over a temperature range that can be used for biological studies, e.g., from about 25° C. to about 45° C. Microbeads can be generated in a range of sizes, but are of sufficient size so that a cell incubated with the microbead will exert force on the microbead, e.g., in response to a stimulus such as a peptide stimulus that induces cellular motion, such as crawling, or induces changes in the cell surface that result in force generation.

In some embodiments, microbeads are prepared using a polymer that has lower critical solution temperature behavior, i.e., the polymer is miscible with the solvent as long as the temperature is kept below the phase transition temperature, while two immiscible phases form above the phase transition temperature. The polymer is thus dehydrated above the transition temperature and when the polymer cooled below the transition temperature, becomes rehydrated (see, e.g. Burmistrova Polymers 3(4):1575-1590, 2011). Accordingly, a microbead produced using such a polymer rehydrates and increases in size upon reducing the temperature to below the phase transition temperature.

In some embodiments, poly(N-isopropylacrylamide) (NIPAM), which has a lower critical solution temperature behavior, can be used as a polymer material for a bead matrix to prepare beads that change diameter with temperature changes. For example, a bead formed from NIPAM can change diameter by about 4% (e.g., from 47 microns to 45 microns between 34° C. and 37° C., which is useful for many biological samples. The Young's modulus of the NIPAM material is well characterized, allowing calibrated 20-30 pN/sec ramping forces to be applied during size shrinking/expansion (see, e.g. Burmistrova Polymers 3(4):1575-1590, 2011). For example, as employed in some embodiments, this approximates the 13-30 pN/s physiological force for T-cell crawling. The polymer may also comprise other materials.

Other thermo-responsive polymers having a lower critical solution behavior can also be used to generate microbeads, e.g., lanthanide-encoded microbeads. Such polymers include those comprising poly(n-vinylamide)s, such as poly(n-vinylcaprolactam); poly(2-alkyl-2oxazoline)s; poly(ether)s; poly(N,N-dimethyaminoethylmethacrylate; and poly(oligo(ethyleneglycol)(methylether)(meth)acrylate)s (see, e.g., Sponchioni et al, Materials Science & Engineering C 102:589-605, 2019).

In some embodiments, a hydrogel comprising a polymer such as poly(acrylic acid)-graft-pi-cyclodextrin (PAAc-g-b-CD)-polyacrylamide (PAAm), that increases in size upon heating (see, e.g., Wang et al., J. Applied Polymer Sci. 111:1417-1425, 2009), can be employed to generate microbeads, e.g., lanthanide-encoded microbeads.

As noted above, the transition temperature of the thermo-responsive polymers can be varied by adjusting the amount of hydrophilic and hydrophobic monomers. Thus, for example, the transition temperature of a polymer having low transition temperature behavior can be increased by increasing the proportion of hydrophilic monomers compared to hydrophobic monomers for copolymerization. Statistical copolymerization with hydrophilic or hydrophobic monomer is a common strategy to tune the transition temperature to a desired value (see, Sponchioni et al., supra).

In some embodiments, suitable polymers for use in preparation of the microbeads is selected from poly(N-alkyl-substituted acrylamide)s (e.g. poly (N-Isopropylacrylamide), poly(N-vinyl amide)s (e.g. N-vinylcaprolactam), poly(2-alkyl-2-2-oxazoline)s, poly(ether)s (e.g. poly(ethylene oxide), poly(propylene oxide)), poly(N,N-(Dimethylamino)ethyl methacrylate), and poly(oligo(ethylene glycol)(methyl ether)(meth)acrylate)s.

In some embodiments, the body and/or the surface polymeric microbeads may also include other components, and in some embodiments, comprise a further agent, such as a polymer coating or a binding moiety, such as a peptide, immobilized to the microbead, as further described in section below. Thus, microbeads as described herein may include or be modified to include one or more reactive functional groups for the attachment of a molecule or molecules, e.g., a peptide, to the surface of the microbead. For example, monomers containing a single acrylate group and a functional group (thiol, amine, hydroxyl, carboxylic acid) can be added before polymerization to yield a microbead with functionality suitable for the attachment of an additional molecule or molecules to the microbeads subsequent to polymerization. The microbeads may include one or more reactive functional groups, e.g., carboxyl groups, hydroxyl groups or amine groups. Functionalization (also referred to as “derivatization”) is additionally described below.

In typical embodiments of the disclosure, the polymeric material includes lanthanide nanoparticles dispersed in the polymeric material to produce lanthanide-encoded microbeads. Preparation of lanthanide-encoded microbeads is detailed below.

### Lanthanide-Encoded Microbeads

The term “lanthanide” refers to elements 57-71 of the periodic table, namely lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). As used herein, “lanthanide” can also refer to combinations of lanthanide elements, compounds containing lanthanide elements or their combinations, or ions containing lanthanide elements or their combinations.

Microbeads spectrally encoded by ratiometric incorporation of lanthanide nanoparticles (Lns), which can be referred to as “spectrally encoded” or “lanthanide-encoded” microbeads, are described, for example, in Nguyen, et al., Advanced Optical Materials 5 (3), 1600548 (2017) and U.S. Pat. No. 10,241,045. Lanthanides have narrow and well-separated emission spectra, making it theoretically possible to generate code sets with 10⁵-10⁶ unique members (Gerver, et al., Lab on a Chip 12:4716-4723, 2012). The ability to produce and discriminate among over 1,100 codes in lanthanide-encoded microbeads is described in (Nguyen et al., 2017, supra).

The microbeads of the present disclosure may include different types of lanthanide nanoparticles. A microbead may include one or more different lanthanide nanoparticles, e.g., 2 or more, 3 or more, 4 or more, 5 or more, 6 or more, 7 or more, 8 or more, 9 or more, or 10 or more, wherein each lanthanide nanoparticle has a different luminescence emission spectrum upon excitation. For example, in some embodiments, the microbeads disclosed herein may include from 1 to 10, from 2 to 10, from 3 to 10, from 4 to 10, from 5 to 10, from 6 to 10, from 7 to 10, from 8 to 10, or from 9 to 10 types of lanthanide nanoparticles, wherein each lanthanide nanoparticle has a different luminescence emission spectrum upon excitation. Signals from the combined luminescence spectra make up the spectral signature of a particular microbead, and are mapped to a unique spectral signature ‘code’ during code deconvolution.

Lanthanide nanoparticles incorporated into microbeads may be prepared using methods such as those described by Xu et al. (Solid State Communications, 2004. 130:465-468), Choi et al. (Journal of Luminescence. 2010. 130:549-553), Wang et al. (Angewandte Chemie International Edition, 2008. 47:906-909), and Nguyen, et al. (2017, supra), the disclosures of which are incorporated by reference herein in their entirety.

Lanthanide nanoparticles according to the present disclosure may be up-converting or down-converting lanthanide nanoparticles. Suitable up-converting lanthanide nanoparticles may include, for example, NaGdF₄: Tm; NaGdF₄: Ln; NaGdF₄Yb; NaGdF₄Er; NaGdF₄Yb, Er; NaYF₄:Er; NaYF₄:Yb; NaYF₄:Er,Yb; NaYF₄:Tm,Yb; LaF₃:Yb,Tm; LaF₃:Yb,Er; and LaF₃:Yb,Ho nanoparticles. Suitable down-converting lanthanide nanoparticles may include, for example, YVO₄:Eu; YVO₄:Dy; and YVO₄:Sm nanoparticles.

In some embodiments, the lanthanides, e.g., the above referenced lanthanides, may be incorporated into the nanoparticles as their respective ions. Materials may be added during preparation of the lanthanide nanoparticles to increase their UV absorption, for down-converters, or IR absorption, for up-converters. For example, in some embodiments, bismuth is incorporated into the lanthanide nanoparticles to increase their UV absorption.

In some embodiments, lanthanide nanoparticles as disclosed herein may be modified (e.g., covered or coated) in a suitable material to facilitate formation of a stable colloid suspension of the lanthanide nanoparticles in a carrier fluid. For example, suitable materials which may be used to cover or coat the lanthanide nanoparticles may include polyethyleneimine (PEI), polyacrylic acid (PAA), sodium citrate, or citric acid. polyethyleneimine (PEI) may be suitable for use, e.g., as a coating.

In some embodiments, production of polymeric microbeads comprising lanthanide nanoparticles includes a step of forming droplets of a fluid comprising a thermo-responsive microbead matrix component, which as described above, can contain hydrophilic and hydrophobic monomers that co-polymerize, and lanthanide nanoparticles; and a second fluid that is immiscible with the first fluid, and a later step of solidifying the microbead matrix component of the formed droplets, thereby forming the polymeric microbeads comprising lanthanide nanoparticles.

In some embodiments, lanthanide-encoded microbeads are producing using, in at least some of the steps, microfluidic devices. Thus, in some instances some steps of the processes for producing lanthanide-encoded microbeads may be performed within a microfluidic device (that is, performed “on chip”) and some steps of the processes for producing lanthanide-encoded microbeads may be performed outside of a microfluidic device used for microbead production (“off chip”). For example, in some embodiments, the fluid comprising a microbead matrix component(s) and lanthanide nanoparticles is prepared “off chip” by mixing the microbead matrix component(s), lanthanide nanoparticles and any other components (if appropriate) outside of the microfluidics device. In some embodiments, the fluid comprising the microbead matrix component(s) and lanthanide particles and the second (immiscible) fluid may be contacted in a microfluidics device to form droplets. Droplet formation within the microfluidics device can be accomplished by various droplet generation methods, active or passive. Generally, droplets of the of the matrix component- and lanthanide nanoparticle-containing fluids are formed in a microfluidic device upon deformation of the interface between the two fluids. Interface deformation is accomplished by using predetermined geometries of flows of the two fluids within the microfluidic device, such as cross-flowing streams and/or flow focusing, in combination of the flow rates and the properties (such as surface tension and viscosity) of the matrix component- and lanthanide nanoparticle-containing fluid (dispersed phase) and the second, immiscible fluid (continuous phase). In some embodiments, a stream of the matrix component- and lanthanide nanoparticle-containing fluid is introduced into of the second, immiscible fluid at one or more intersections of channels within the microfluidic device. For example, the matrix component- and lanthanide nanoparticle-containing fluid may be flowing through a first channel, and be introduced into a stream of second fluid flowing through a second channel at a T-junction of the first channel and the second channel (so-called droplet generation with T-junction), with the droplets of the matrix component- and lanthanide nanoparticle-containing fluid being formed in a stream of the second fluid in the channel downstream of the T-junction (which can be referred to as “droplet channel”). In another example, the matrix component- and lanthanide nanoparticle-containing fluid may be flowing through a first channel, and be introduced into streams of second fluid flowing from opposing sides of a cross-channel (second channel), with the two streams of the second fluid surrounding the stream of the first fluid and forcing droplet formation into an “output” or “droplet” channel. The above configuration is typically used in a so-called “droplet generation by flow focusing,” and can employ one or more flow-focusing components, such as constrictions or flow-focusing nozzles. It is to be understood that the above examples of droplet formation mechanisms are exemplary only and non-limiting, and other ways to generate droplets may be employed. Droplet size may be changed by changing the pressure and/or the flow speed of the first and the second fluids and/or by adjusting the geometry of the microfluidic device channels.

In some embodiments of the methods of preparing lanthanide-encoded polymeric microbeads, the formed droplets are removed from the microfluidic device. For example, removal of the formed droplets from the microfluidic device can be accomplished by the flow of the second fluid with the formed droplets through one or more droplet outlet channels, with the droplets exiting the microfluidics device through one or more droplet outlets. In the embodiments involving removal of the formed droplets from the microfluidics device, microbead matrix component of the formed droplets is solidified outside of the device (“off chip”), thereby forming the polymeric microbeads comprising lanthanide nanoparticles. However, some embodiments of the processes for producing lanthanide-encoded microbeads may not include removal of the formed droplets from the microfluidics device, and solidification of the droplets may be accomplished within the microfluidics device (“on chip”), in which case the polymeric microbeads comprising lanthanide nanoparticles are formed within the microfluidics device and removed from the microfluidics device through appropriate outlet channels and/or outlets.

The thermo-responsive polymer used in forming the microbeads are polymerized using known techniques. For example, in some embodiments polymerization is accomplished by exposure to an appropriate wavelength of light, such as ultraviolet (UV) light. Other components, such as a cross-linker, a polymerization initiator, or a catalyst may also be employed. For example, UV-polymerization involves irradiating the thermo-responsive matrix polymer, e.g., droplets as described above, with UV radiation to polymerize a polymerizable microbead matrix component, the first fluid may include a photoinitiator. A suitable photoinitiator may include a compound that, when exposed to UV light, undergoes a photoreaction, producing reactive species that are capable of initiating polymerization. Exemplary photoinitiators include, but are not limited to, acetophenones, benzyl and benzoin compounds, benzophenone, cationic photoinitiators, and thioxanthones. In another example, chemical polymerization involving a polymerization catalyst may be used. For instance, chemical polymerization of acrylamide or acrylamide derivatives may be used, in which case ammonium persulfate (APS) may be included in the matrix component-containing and lanthanide nanoparticle-containing fluid and tetramethylethylenediamine (TEMED) may be included in the second, immiscible fluid. TEMED and ammonium persulfate are both polymerization catalysts, with TEMED diffusing into the matrix-component containing particle upon contact with the second, immiscible fluid and accelerating the rate of release of free radicals from APS, which, in turn catalyze the polymerization.

Lanthanide nanoparticles included in the lanthanide-encoded microbeads may be modified (for example, covered or coated) in a suitable material to facilitate formation of a stable colloid solution of the lanthanide nanoparticles. Suitable materials may include materials preventing aggregation of the lanthanide nanoparticles and/or facilitate maintenance of a nanoparticle form of the lanthanide nanoparticles. For example, suitable materials that may be used to cover or coat the lanthanide nanoparticles suspended in a hydrophilic fluid may include polyethyleneimine (PEI), polyacrylic acid (PAA), sodium citrate, or citric acid. PAA may be used as a coating material to enhance the photostability of the lanthanide nanoparticles, in addition to facilitating stable colloid formation. In another example, lanthanide nanoparticles may be coated with polyethylene glycol diacrylate (PEGDA) 200 or PEGDA 250 to facilitate formation of a colloidal solution in a hydrophobic fluid. In a further example, lanthanide nanoparticles may be coated with poly(propylene carbonate), poly(ethylene succinate), or poly(vinyl chloride) carboxylated to facilitate forming a colloidal solution in a hydrophobic fluid.

Some methods of producing lanthanide-encoded polymeric microbeads advantageously allow for inclusion of reactive (or functional) groups on a surface of the polymeric microbeads during microbead solidification, i.e., the lanthanide-encoded polymeric microbeads can be functionalized as a part of the production process without adding additional steps after microbeads are performed. Functionalization of lanthanide-encoded polymeric microbeads can be accomplished by including a suitable amphipathic compound in a second, immiscible fluid used for droplet generation. Such a suitable amphipathic compound is capable of covalently bonding with the microbead matrix component during the solidification step. A suitable amphipathic compound includes one or more reactive groups that remain free after covalent binding of the amphipathic compound to the surfaces of the polymeric microbeads, and these free reactive groups can be used for subsequent attachment of molecules or moieties of interest to the microbeads, e.g., a binding moiety. Molecules of the suitable amphipathic compound included in the continuous phase are driven to and remain at the interface of the immiscible fluid and the matrix component- and lanthanide particle-containing fluid after droplet formation, with the hydrophobic parts of the amphipathic molecules facing a hydrophobic fluid (which may be the matrix and nanoparticle-containing fluid or the immiscible fluid), and the hydrophilic parts of the amphipathic molecules facing a hydrophilic fluid (which may be the matrix and nanoparticle-containing fluid or the immiscible fluid). A suitable amphipathic compound, or compounds (if more than one is used), is included at a concentration sufficiently low (for example, from about 0.002% to 2% w/w or v/v) to limit cross-linking of the molecules of the amphipathic compound with the molecules of the microbead matrix component to the surface of the microbeads during their solidification.

An amphipathic compound used for microbead functionalization includes one or more reactive groups that remain free upon the covalent attachment of the amphipathic compound to the surface of the polymeric microbeads during the solidification step of the microbead production process. As discussed above, the reactive (or functional) groups may be used for subsequent covalent coupling of a molecule or moiety of interest. The reactive (or functional) group can be or comprise a carboxyl group, an amino group, an azide group, a hydroxyl group, a hydrazide group or a chloromethyl group. The polymeric microbeads can be functionalized by two or more different reactive groups during their production by using, for example, an amphipathic compound with two or more reactive groups. In another example, multiple amphipathic compounds with different reactive croups can be added to the continuous phase during droplet generation. Some non-limiting examples of the amphipathic compounds that can be used for microbead functionalization are: for functionalization with carboxyl groups, unsaturated fatty acids, such as 10-undecenoic acid, 4-pentenoic acid, 5-hexenoic acid, 6-heptenoic acid, 7-octenoic acid, 8-nonenoic acid, or 9-decenoic acid; for functionalization with amino groups, amphipathic amines, such as pent-4-enylamine, N-(3-Aminopropyl)methacrylamide, 2-Aminoethyl methacrylate, or N-(2-aminoethyl) methacrylamide; for functionalization with azide groups, 3-azidopropyl acrylate or 3-azidopropyl methacrylate; for functionalization with hydroxyl groups, 4-Penten-1-ol; for functionalization with hydrazide groups, hydrazido acrylate; for functionalization with chloromethyl groups, chloromethyl acrylate.

In some embodiments, functionalized microbeads be produced using, as starting materials, a hydrophilic fluid comprising a microbead matrix component (which may include multiple components). The fluid may be hydrophilic due to hydrophilic properties of the microbead matrix component and/or to a presence of a hydrophilic solvent, such as water, polar protic solvents, or water-polar protic solvent mixtures. Exemplary polar protic solvents include, but are not limited to, acetic acid, methanol, ethanol, n-propanol, or n-butanol.

In some embodiments, the microbead matrix component may, by itself, be a hydrophilic liquid monomer (for example, N-alkyl-substituted acrylamides (e.g. N-Isopropylacrylamide, N-vinyl amides (e.g. N-vinylcaprolactam), 2-alkyl-2-2-oxazolines, ethers (e.g. ethylene oxide, propylene oxide), N,N-(Dimethylamino)ethyl methacrylate and oligo(ethylene glycol)(methyl ether)(meth)acrylates.

In some other embodiments, the first fluid can be a hydrophilic solution of a microbead matrix component, such as an aqueous solution (an aqueous fluid comprising a matrix component, for example, a polymerizable component). In some embodiments, a microbead matrix component may contain one or more suitable synthetic thermo-responsive polymers including, but not limit to, poly(N-alkyl-substituted acrylamide)s (e.g. poly (N-Isopropylacrylamide), poly(N-vinyl amide)s (e.g. N-vinylcaprolactam), poly(2-alkyl-2-2-oxazoline)s, poly(ether)s (e.g. poly(ethylene oxide), poly(propylene oxide)), poly(N,N-(Dimethylamino)ethyl methacrylate) and poly(oligo(ethylene glycol)(methyl ether)(meth)acrylate)s. Such polymers exhibit a phase transition temperature that results in a marked change in solubility.

In some embodiments, the polymer component-containing fluid may be contacted with an immiscible fluid comprising an amphipathic compound. Some non-limiting examples of suitable components of the hydrophobic continuous phase fluids are oils, such as a mineral oil or a fluorinated oil, liquid hydrocarbons, liquid fatty acids, siloxanes or fluorocarbons. In some embodiments, a hydrophobic second fluid can be a mineral oil comprising a surfactant (for example, Abil EM90 and/or Span 80), dioctyl phthalate comprising a surfactant (for example, Tween 20), oleic acid comprising a surfactant (for example, Tween 20), perfluorinated Fluorinert FC-40 comprising a surfactant (for example, Zonyl® FSO), or octamethyltrisiloxane comprising a surfactant (for example, Triton X-100). A surfactant can be present at a concentration of from about 0.01% to about 5%, v/v or w/w.

Some non-limiting examples of the amphipathic compounds that can be used for microbead functionalization and included in the second fluid are: for functionalization with carboxyl groups, unsaturated fatty acids, such as 10-undecenoic acid, 4-pentenoic acid, 5-hexenoic acid, 6-heptenoic acid, 7-octenoic acid, 8-nonenoic acid, or 9-decenoic acid; for functionalization with amino groups, amphiphathic amines, such as pent-4-enylamine, N-(3-Aminopropyl)methacrylamide, 2-Aminoethyl methacrylate, or N-(2-aminoethyl) methacrylamide; for functionalization with azide groups, 3-azidopropyl acrylate or 3-azidopropyl methacrylate; for functionalization with hydroxyl groups, 4-Penten-1-ol; for functionalization with hydrazide groups, hydrazido acrylate; for functionalization with chloromethyl groups, chloromethyl acrylate. The polymeric microbeads can be functionalized by two or more different reactive groups during their production by using, for example, an amphipathic compound with two or more reactive groups. In another example, multiple amphipathic compounds with different reactive croups can be added to the continuous phase during droplet generation.

In some embodiments, microbeads are functionalized to include reactive groups such as carboxyl groups, amino groups, azide groups (which can be used for “click” chemistry), hydroxyl groups (which can be used for cyanogen bromide-activated coupling of proteins), and hydrazide groups (which can be used for oxidized carbohydrate proteins) and chloromethyl groups (which can be used for coupling of NH₂ groups in protein or other biological molecules) (47).

### Determining Lanthanide Spectral Signatures of Spectrally Encoded Microbeads

The combined luminescent signals generated from lanthanide nanoparticles provide a unique pattern, i.e., signature, that is specific to a microbead. In some embodiments, each of the lanthanide spectral signatures comprises a europium (Eu) signal, a dysprosium (Dy) signal, a samarium (Sm) signal, a cerium (Ce) signal, a terbium (Tb) signal, a lanthanum (La) signal, a praseodymium (Pr) signal, a neodymium (Nd) signal, a gadolinium (Gd) signal, a holmium (Ho) signal, an erbium (Er) signal, a thulium (Tm) signal, an ytterbium (Yb) signal, or a combination thereof. In some embodiments, each of the microbeads comprises a plurality of lanthanide nanoparticles. In some embodiments, the lanthanide nanoparticles comprise a lanthanide-doped host lattice. In some embodiments, the host lattice is yttrium orthovanadate, lanthanum phosphate, or a combination thereof. In some embodiments, each of the microbeads further comprises a polymer coating covering the lanthanide nanoparticles, and wherein the capture oligonucleotides are covalently bonded to the polymer coating. In some embodiments, a plurality of the microbeads contains at least 100 lanthanide spectral signatures. In some embodiments, the plurality of microbeads contains at least 1000 lanthanide spectral signatures.

In general, each of the spectral signatures employed in an assay contains signals generated from predetermined amounts of two or more lanthanides (e.g., two or more Eu-, Dy-, Sm-, Ce-, Tb-, La-, Pr-, Nd-, Gd-, Ho-, Er-, Tm-, Yb-containing materials such as nanoparticles). Lanthanide materials in the microbeads can be excited with UV light (e.g., 275 nm or 292 nm) and emitted luminescent signals can be detected in the range of 400-800 nm (e.g., 435 nm, 474 nm, 527 nm, 536 nm, 546 nm, 572 nm, 620 nm, 630 nm, 650 nm, or 780 nm). A signal in a spectral signature may be measured as an absolute value or as a ratio of the signal to another reference signal. As a non-limiting example, a set of unique spectral signatures can be prepared with microparticles that contain europium-dope yttrium orthovanadate (YVO₄:Eu) to generate a reference signal and varying amounts of YVO₄:Dy, YVO₄:Sm, YVO₄:Tm, and LaPO₄:CeTb.

In an array configuration as described herein, a lanthanide spectral signature of a microbead is typically determined via deep UV imaging as described above of the compartments of the arrayed configuration. The spectral signature of the microbeads present in the cell analysis compartments and the spatial position of those compartments in the array can thus be determined. In some embodiments, lanthanides embedded in different host matrices excite at low energy light, e.g., 980 nm IR, and emit in visible light. In applications employing such “up-converting” lanthanide:host matric combinations, the spectral signature is determined via infrared imaging.

Assays to Assess Force-Elicited Cellular Responses Mediated by a Binding Moiety that Targets a Cell Surface Molecule

In some embodiments, thermo-responsive microbeads comprising a lanthanide spectral signature as used in the methods described herein are used to evaluate a force-mediated cellular response in cells incubated with the microbeads, or the ability of a moiety to elicit a force-mediated response. As used herein, a “force-mediated cellular response” refers to a change in a cellular phenotype that occurs in response to mechanical force exerted against the surface of a cell. In some embodiments, a binding moiety to be evaluated is immobilized to the surface of a single spectrally encoded microbead that has a spectral identifier that differs from the spectral identifiers of microbeads to which different binding moieties are immobilized. A binding-moiety that is “immobilized” to the microbead means that the binding moiety is attached to the microbead and doesn't detach from the microbead during incubation with cells or through heating and cooling steps. “Determining that a binding moiety is associated” with a microbead means that the binding moiety attached to the microbead can be identified based on the spectral signature of the microbead.

An “arrayed configuration” (also referred to herein an “array configuration”) as used herein refers to a collection of single compartments, at least a portion of which are analysis compartments in which each contains at least one cell and a microbead co-compartmentalized with the cell. In some embodiments, at least a portion of the collection of single compartments each contain a single cell and a single microbead co-compartmentalized with the single cell. An array configuration may be an “ordered array” in which the compartments are addressable and can be assigned to known locations.

A “compartment” as used herein refers to any partially or fully enclosed space that separates one single cell/microbead from another. Thus, a compartment can include a microwell, a droplet, a micropore, a microfluidic chamber, and the like.

In some embodiments, a population of binding moieties is evaluated for the ability to stimulate a force-induced cellular response. In some embodiments, an array is generated in which spectrally encoded microbeads are distributed to individual compartments in an array configuration (e.g., a microwell) where each of the microbeads has a different binding moiety attached thereto. In some embodiments, single cells can be distributed to the compartments for analysis. In a single cell analysis, ideal distributions to obtain a plurality of wells containing one cell and one bead each, nearly all wells containing both microbeads and cells will have different spectral code assignments. In some embodiments, multiple cells, e.g., 2, 3, 4, or 5, are loaded into a single well, e.g., to increase the number of cells analyzed in a single assay. Thus, for example, 1, 2, 3, or 4 cells can be loaded into a single well for incubation with a single microbead. The temperature of the array is then changed so that the beads increase in size, e.g. thermos-responsive microbeads prepared with a polymer that has lower critical solution behavior will increase in size when the temperature is adjusted to below the phase transition temperature. The increase in size results in the application of force to the cells and thus binding moieties tethered to the microbeads that bind to a mechanosensing molecule on the surface can be evaluated for the ability to elicit cellular responses via the mechanosensing molecule. Any number of assays to measure cellular responses can be assessed. For example, such an assay can comprise use of a signal-generating reagent, e.g., a detection reagent labeled with a detectable label, such as a fluorescent dye, chemiluminescent agent, radioisotope, or other label. In some embodiments, a cellular response is assessed by image analysis without the use of a detectable reagent. In some embodiments, cellular perturbations induced by the binding moiety may be evaluated, e.g., phenotypic changes, including changes in morphology and the like, may be assessed.

Each compartment of an array that comprises a cell and microbead is assessed to determine whether a signal is generated. The spectral signature of the microbead in each compartment is also determined. Signal detection and spectral signatures can be determined at the same time, or sequentially, in any order. The spectral code associated with a compartment that generates a signal can then be used to determine the binding moiety immobilized to the bead in that compartment that elicited the signal.

### Binding Moieties

Generally, the binding moiety immobilized on a thermos-responsive microbead targets a molecule on the surface of a cell. For example, in some embodiments, the binding moiety targets an adhesion molecule on the surface of a cell, e.g., an integrin or cadherin. In some embodiments, the binding moiety targets an immune cell, e.g., a T-cell receptor or B-cell receptor on the surface of a T-cell or B-cell, respectively. In some embodiments, the binding moiety may target a receptor on the surface of a platelet, e.g., glycoprotein 1b-IX. In some embodiments, a binding moiety may target a glycosaminoglycan, proteoglycan, phospholipid, or any other cellular membrane component. In some embodiments, the binding moiety may target intracellular mechanosensing biomolecules such as motor proteins (myosin, kinesin and dynein). Thus, in some embodiments, force due to the size change of thermo-responsive microbeads can be applied to the motor protein to induce assist/stall force to motor proteins.

Binding moieties can be any type of molecule that targets a molecule on the surface of a cell. Such target molecules include, e.g., a cell surface receptor. In some embodiments, the binding moiety is a polypeptide. In other embodiments, the binding moiety may be a carbohydrate, lipid, or other molecule that can interact with the cell surface molecule. In the context of a binding moiety immobilized to a microbead, the “density” of the binding moiety refers to the number of molecules immobilized to the microbead. The number of molecules may be expressed as the number bound per microbead, or number bound per unit area of the microbead.

In some embodiments, pools of thermo-responsive microbeads having different spectral signature can be evaluated with a plurality of cells, e.g., cells layered onto a layer of microbeads in a compartment. A signal generated from one or more cell in a compartment can then be associated with the spectral signatures of the microbeads in the compartment and further evaluated, e.g., on a single-cell level.

Force-induced cellular response in cells from many sources can be evaluated, including both plant and animal cells. In some embodiments, mammalian cells, e.g., human cells, are evaluated. In some embodiments, the cells are B-cells, T-cells, cells that response to stretch, such as muscle cells, cancer cells, stem cells, neurological cells, peripheral blood mononuclear cells, lymphocytes, or cells from a cell line. In some embodiments, the cells are obtained from a tissue e.g., a human tissue. In some embodiments, single cells from transgenically modified organisms may be evaluated. In some embodiments, the cells range in diameter from 1 to 100 m, with the corresponding thermo-responsive beads ranging in diameter from 10 μm to 200 μm. As described herein, the force applied to the potential cells in contact with the beads can be tuned by varying the rigidity of the bead matrix, the rate of size change of the bead, and the rate of temperature change or temperature range to which beads are heated and cooled.

In some embodiments, thermo-responsive spectrally encoded microbeads as described herein are employed to evaluate peptides that activate immune system cells, such as T-cells or B-cells. Methods of evaluating peptides to activate T-cells are further detailed below. One of skill understands that force-induced responses in B-cells or other cell types can be determined using parallel methodology.

### Peptide-MHC Complexes

Candidate peptides from protein antigens, e.g., cancer antigens, viral antigens, bacterial antigens, parasitic antigens, autoimmune antigens, or any other antigen of interest that are assessed for the ability to mediate force-induced T cell responses, e.g., T cell activation, can be selected based on various criteria, including sequence motifs, peptide length, and similarity to known T-cell epitopes. In the present application, such candidate MHC epitope peptides are presented as part of a major histocompatibility complex.

Candidate peptides related to certain T-cell receptors can be predicted by established algorithms (see, e.g., Ogishi & Yotsuyanagi Front. Immunol. 10, Article 827, 2019; Chen et al, Nature Biotechnol 37:1332-1343, 2019; and Blood 134 (Supplement 1): 84, 2019) or experimental pMHC tetramer binding, or cell co-culture experiments.

As used herein, “peptide-loaded MHC,” “pMHC,” and “peptide-loaded MHC complex” are used interchangeably to refer to a major histocompatibility complex protein loaded with a peptide in the antigen binding groove. The term includes multimeric forms, e.g., tetramer complexes, in which four MHC molecules are each loaded with the peptide of interest. The term “MHC” as used herein refers to major histocompatibility complex polypeptides from any animal, including humans. Thus, “MHC” as used herein includes reference to human leukocyte antigen (HLA) polypeptides. In some embodiments, the MHC complex protein may be an MHC Class I (MHC I) complex that associates with CD8+ T cells. In other embodiments, the MHC complex may be an MHC Class II (MHC II) complex that associates with CD4+ T cells. In humans, HLA Class I molecules include HLA-A, HLA-B, and HLA-C molecules, such as various HLA-A1, HLA-A2, HLA-A3, HLA-A24, HLA-B07, HLA-B08, HLA-B27, HLA-B44, HLA-B58, HLA-B62 alleles, or HLA-C MHC Class I alleles. The six major HLA class II molecules include HLA-DPA1, HLA-DPB1, HLA-DQA1, HLA-DQB1, HLA-DRA, and HLA-DRB1 alleles.

In some embodiments of the present invention, the pMHC is a tetramer complex of four MHC molecules each loaded with a peptide comprising a candidate T cell epitope. pMHCs comprising different peptides are immobilized to individual thermo-responsive spectrally-encoded microbeads, e.g., generated using NIPAM as the polymer or an alternative polymer that has lower critical solution behavior, each having a spectral signature that differs from other microbeads, such that there is generally a 1:1 relationship between the peptide species and bead spectral signature. In some embodiments, pMHC are attached to the microbeads via biotin-streptavidin interactions. Thus, for example, biotin-modified pMHC may be loaded onto the microbeads via interaction with streptavidin coated on the surface of the spectrally encoded microbeads. pMHC are typically loaded at a low density that reflects physiological pMHC density, for example at a density of about 0.05 pMHC/μm² (which provides 3-4 molecules/cell). Beads are distributed into individual compartments, e.g. a microwell array. T cells are then distributed to the compartments. In some embodiments, a single T cell is compartmentalized with a single bead. In some embodiments, e.g., the array is heated from room temperature to 37° C. and then cooled to 34° C. In this example, cooling results in an increase in microbead size, thus exerting force on the cell. Heating and cooling time frames are determined by the assay used to evaluated T cell activation. For example, in some embodiments, microbeads may be heated for a short time, e.g., one minute, and cooled for another relatively brief period of time, e.g., two minutes, and a parameter that changes quickly and reflects T-cell activation, such as calcium flux, is measured following cooling, e.g., at three minutes, using an assay such as image analysis. In alternative embodiments, other markers of T cell activation, such as upregulation of CD69 or CD137 (e.g., measured after 12-14 hours), or LCK phosphorylation (e.g., measured after one minute), can be evaluated. Thus, for example, heating can be employed for a time frame, such as one minute or longer, and followed by longer cooling time periods, e.g., self-cooling over time and expression of CD69 or CD137 can be assessed. In some embodiments, T cells may be loaded into compartments containing the microbeads that are at the incubation temperature, e.g., an incubation temperature of 37° C. The array is then cooled to the desired temperature, e.g., 34° C., that crosses the phase transition temperature of the thermos-responsive polymer component of the microbead.

### Analysis of the Amount of Force Exerted on Cells

In some embodiments, the effects of different levels of force on force-induced responses can be evaluated. For example, in some embodiments, the amount of force generated by a given microbead on a cell of a particular size can be determined by an equation:

$$F = \frac{2 {R}_{cell}}{\sqrt{6}} \times \frac{K}{\sqrt{{R}_{bead}}} \times Δ {R}_{bead}^{1.5}$$

This can be validated by measuring rigidity of beads at different temperatures using techniques such as atomic force microscopy. In an illustrative embodiment, rigidity is measured by a parallel plate rheometer. For example, a thin pNIPAM slab (D=−35 mm) is made with the same ingredient as the thermos-responsive microbeads employed in the analysis. The slab is placed below a 40.0 mm parallel plate under solvent sealed chamber at room temperature. A frequency sweep is performed with strain and angular frequency at 2.0% and 10.0 rad/s, respectively. The shear modulus/rigidity can be calculated by combining the storage modulus (G′) and loss modulus (G″) and averaged during the 60 sec measurement.

The beads and pMHC concentrations may also be varied to provide multiplex reactions in which different forces are generated by T-cell incubation with beads and/or different densities of pMHC are employed. For example, use of different amounts of sodium acrylate (or any other hydrophilic monomer), can provide beads having different phase transitions temperatures and therefore different force responses. For example, the following amounts of sodium acrylate employed as a co-polymer with 9.4% (w/v) NIPAM can produce forces as follows:
50 mM sodium acrylate: 40-60 pN/s
55 mM sodium acrylate: 20-30 pN/s
60 mM sodium acrylate: 10-15 pN/s

Lanthanide codes may also be linked with different pMHC densities on the beads. For example, a spectral code of a microbead can be linked to density that provides a concentration of 3-4 pMHCs/cell, a second spectral code can be linked to a density that provides a concentration of 30-40 pMHC/cell, and a third spectral code can be linked to a density that provides a concentration of 150-200 pMHCs/cell.

It is understood that the examples and embodiments described herein are for illustrative purposes only and that various modifications or changes in light thereof will be suggested to persons skilled in the art and are to be included within the spirit and purview of this application and scope of the appended claims.

### Examples

### Preparation of Heat-Responsive Beads

A flow focusing microfluidic device was employed to form aqueous polymer droplets within a fluorinated oil stream (HFE7500 with 2% (w/w) ionic Krytox and 0.05% (v/v) acrylic acid), which can be collected and subsequently polymerized into hydrogel beads via UV illumination. Specifically, NIPAM (9.4% w/v), poly(ethyleneglycol) diacrylate (PEG-DA) (0.15% v/v), 1M sodium acrylate (NaAc) solution (pH=7 in DI water, 3.5% v/v), and lithium phenyl-2,4,6-trimethylbenzoylphosphinate (LAP) solution (39.2 mg/ml in DI water, 2.5% v/v) with unique ratios of lanthanide nanophosphors (LNs: Eu, Sm, Tm, Dy, and CeTb) were mixed together as the aqueous/dispersed phase. The LNs were synthesized as described (e.g., U.S. Pat. No. 10,241,045). Parallel flow focuser chips were fabricated in Polydimethylsiloxame (PDMS) using standard SU-8 soft lithography. Aqueous phase with ratiometric LNs and HFE7500 with Ionic Krytox (IK) and acrylic acid (AAc) were introduced into the aqueous inlet and oil inlet, respectively, yielding high-throughput production of pre-gel droplets at the flow-focusing nozzle. After droplet formation, 2 minutes of UV polymerization (IntelliRay, ˜100 mW/cm²) with full power was carried out to crosslink the droplets. The AAc in the oil phase will gradually diffuse into the aqueous phase to form a carboxy shell for the subsequent functionalization, where streptavidin can be crosslinked to the bead surface using a carbodiimide crosslinker strategy (EDC chemistry).

Loading of Peptides onto the Beads

To load candidate peptides into peptide MHC (pMHC) complexes, UV-cleavable peptide-MHC molecules with a biotin tag were used, followed by UV-facilitated peptide exchange to obtain the target pMHC molecules. The biotin-pMHCs were then incubated with the streptavidin coated smart beads, establishing a 1:1 relationship between the peptide species and bead LNs code. To obtain a physiological low pMHC density, ˜100 pM pMHCs were incubated with ˜300,000 beads in 500 μl solution, yielding ˜3 to 4 pMHCs across a 100 μm² area.

NIPAM (9.4% w/v), poly(ethylene glycol) diacrylate (PEG-DA) (0.15% v/v), 1M sodium acrylate (NaAc) solution (pH=7 in deionized (DI) water, 3.5% v/v), and lithium phenyl-2,4,6-trimethylbenzoylphosphinate (LAP) solution (39.2 mg/ml in DI water, 2.5% v/v) with unique ratios of lanthanide nanophosphors (such as LNs:Eu, Sm, Tm, Dy, and CeTb) were mixed together as the aqueous/dispersed phase. The LNs were synthesized as described (e.g., Nguyen et al., Advanced Optical Materials 5(3):1600548, 2018. Parallel flow focuser chips were fabricated in polydimethylsiloxame (PDMS) using standard SU-8 soft lithography. Aqueous phase with ratiometric LNs and HFE7500 with Ionic Krytox (IK) and acrylic acid (AAc) were flowed into the aqueous inlet and oil inlet, respectively, yielding high-throughput production of pregel droplets at the flow-focusing nozzle. After droplet formation, 2 mins UV polymerization (IntelliRay, ˜100 mW/cm²) with full power was carried out to crosslink the droplets. The AAc in the oil phase will gradually diffuse into the aqueous phase to form a carboxy shell for the subsequent functionalization, where streptavidin can be crosslinked to the bead surface using carbodiimide cross-linker strategy (EDC chemistry).

Functionalized ‘smart’ beads were then be collected for subsequent bright field and fluorescence imaging, as well as on an indium tin oxide (ITO) glass slide designed to provide temperature control at a range from 25° C. to 37° C. to quantify temperature response. The availability of streptavidin was checked by incubating 0.5 μL 1 mg/mL Biotin-Atto647 and ˜400 μL bead slurry (1 million/mL) for 1 h at room temperature, followed by 3 times PBST washing. Both streptavidin functionalized and bare ‘smart’ beads were used and compared at the Atto647 channel.

Quantitatively probing the relationship between force-dependent pMHC-TCR binding and subsequent T cell activation in high-throughput requires the ability to monitor time-dependent activation for many pMHC-interacting cells in parallel. To accomplish this, we loaded encoded smart beads bearing different pMHC sequences together with T cells in a 1440 microwell array made by PDMS (50 μm diameter×56 μm deep, resulting in ˜98% well occupancy) and then visualize T cell calcium flux induced by the thermo-stimulated expansion force to quantify activation. To facilitate rapid heating and cooling, microwell arrays are mounted on indium tin oxide (ITO) glass, which is transparent and changes temperature in response to applied current. The assay is conducted as follows: (1) microwells are loaded with smart beads, (2) microwells are loaded with T cells and incubated to allow T cell attachment to beads, (3) the microwell device is heated (to 37° C.) and then cooled (to 34° C.) to apply shear force to bead-attached T cells via smart bead expansion, and (4) T cells stained with calcium-sensitive dye (e.g. Cal-520) are imaged continuously for real-time monitoring of T cell activation by quantifying calcium flux. For assays in which spectrally encoded beads are used to profile responses to multiple different pMHCs, smart beads can be imaged in the lanthanide channels, e.g., after calcium imaging, to read embedded spectral codes and identify pMHC sequences displayed on each bead. Force multiplexing and concentration multiplexing assays follow the same experimental procedures.

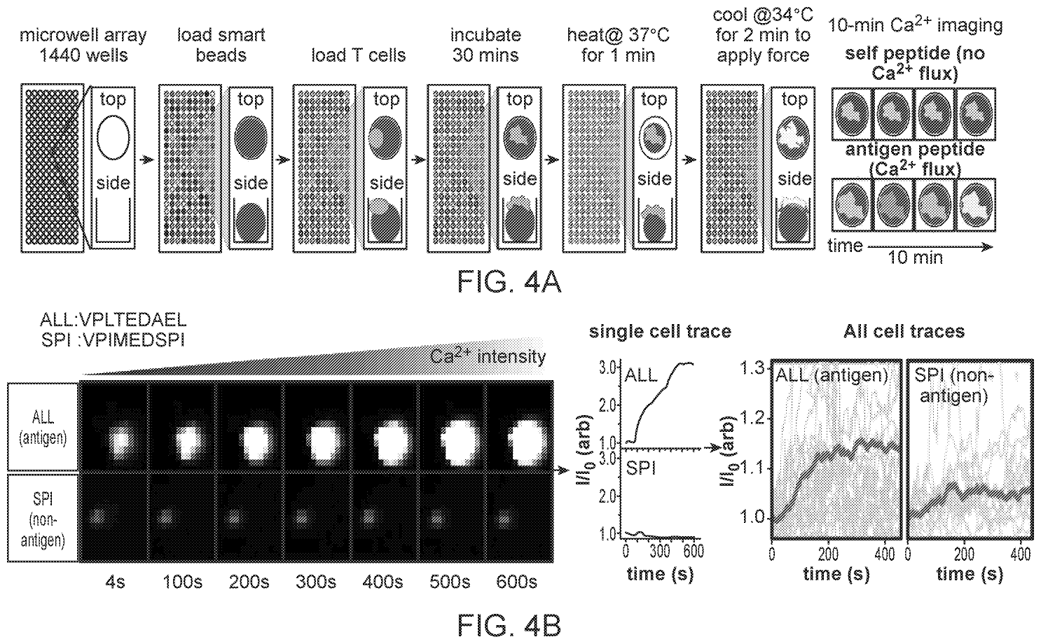

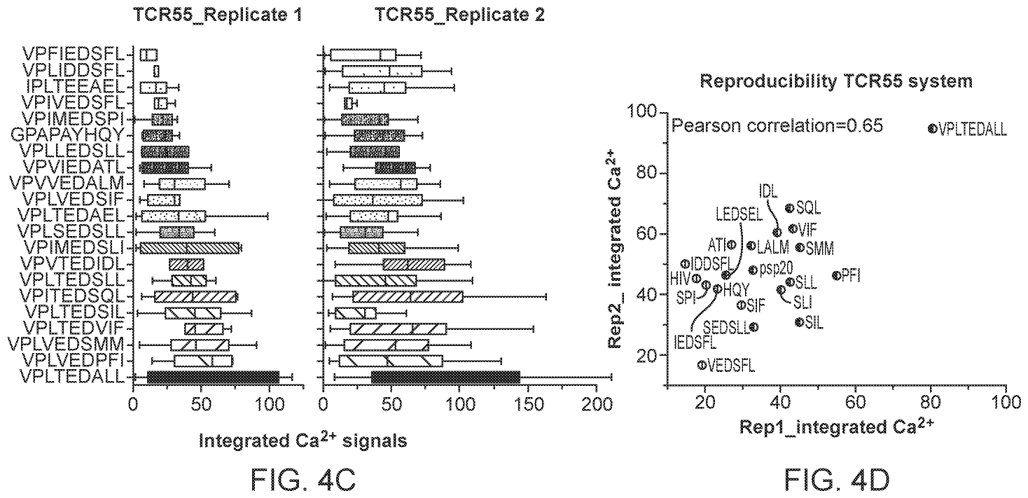

An experiment (FIG. 4A) was designed to initially evaluate peptides (21 peptides in total) in which antigenic peptides that elicit T cell activation in vivo are expected to show force-dependent activation for true antigenic pMHCs, an absence of force-dependent activation for self pMHCs, and high-affinity yet non-stimulatory pMHC. In this illustrative assay, 21 varieties of encoded smart beads displaying stimulatory pMHCs and nonstimulatory pMHCs were paired with TCR55 T cells. SKW3 T cells transfected with TCR55 TCR were maintained in RPMI 1640 GlutaMAX (Sigma-Aldrich) supplemented with 10% (v/v) fetal bovine serum (FBS) (Sigma-Aldrich), 100 U/ml penicillin, 100 U/ml streptomycin (Life technology), and reached the log phase of growth prior to performing the triggering experiment. Microwell arrays containing 1440 wells were fabricated in polydimethylsiloxame (PDMS) using standard SU-8 soft lithography (23). The diameter, height of each well, and center-to-center distance between neighbor wells were 50 m, 56 μm and 50 m, respectively. On the day preceding the experiment, a new device was filled with wash buffer (PBST, 0.1% (v/v) Tween-20 (Sigma-Aldrich)) and stored in a humid chamber (a pipette tip box half-filled with water) overnight. pMHC coated beads were obtained by mixing 0.5 μL 10 nM biotin-pMHC and ˜30,000 streptavidin coated ‘smart’ beads to a final volume to 56 μL in PBST buffer and incubating for 1 h at room temperature, followed by 3 times PBST washing. For the concentration multiplexing experiment, 5 μL or 25 μL 10 nM biotin-pMHC were added. The functionalized beads were then re-suspended into 20 pL wash buffer and flowed into the chip at 20 μL/h. After the bead-loading, the chip was then washed with colorless RPMI 1640 (SigmaAldrich) for Cal-520 (AAT Bioquest) stained T cell loading. The Ca²⁺ flux dye loading procedure is identical to the previous method (11). The cells were flowed into the chip at ˜ 20 μL/h to minimize the drag force applied between the bead and cell. After 30-min incubation at room temperature, the chip was placed on the 37° C. ITO glass for 1-min heating and then cooled to 34° C. for 2 min, during which the expansion force was applied to the possible TCR-pMHC interaction. Then the cells were excited using a 488-nm laser to monitor Ca²⁺ sensitive Cal-520 fluorescence every 4 sec. After 10 mins, the Ln signals were recorded. The temperature was lowered to apply expansion force and the cells were then imaged by measuring fluorescence at 488 nm to monitor Ca²⁺ flux.

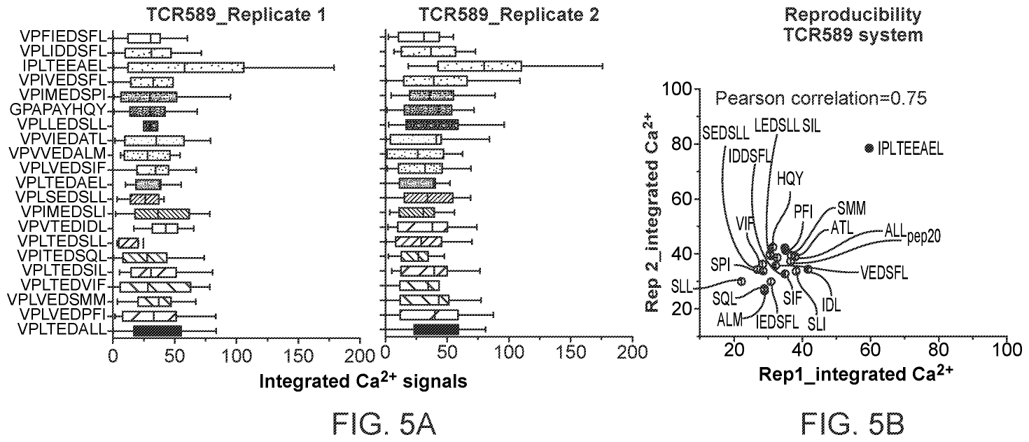

These results established that TCR55 T cells bound to stimulatory (VPLTEDALL) pMHC-coated smart beads (FIG. 4B-4D) showed increased Ca²⁺ flux upon expansion, whereas nonstimulatory peptides (the rest of 20 peptides) failed to induce enough Ca²⁺ ions. However, for TCR589 T cells (FIG. 5A-5B), a peptide having the sequence IPLTEEAE exhibited the maximum Ca²⁺ response after force application.

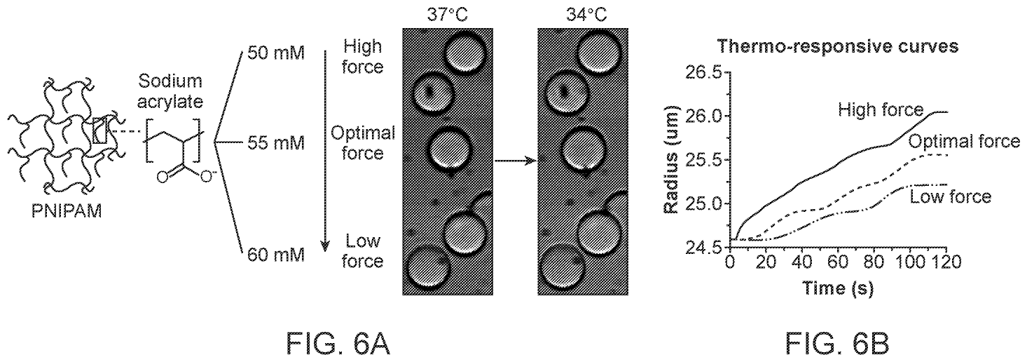

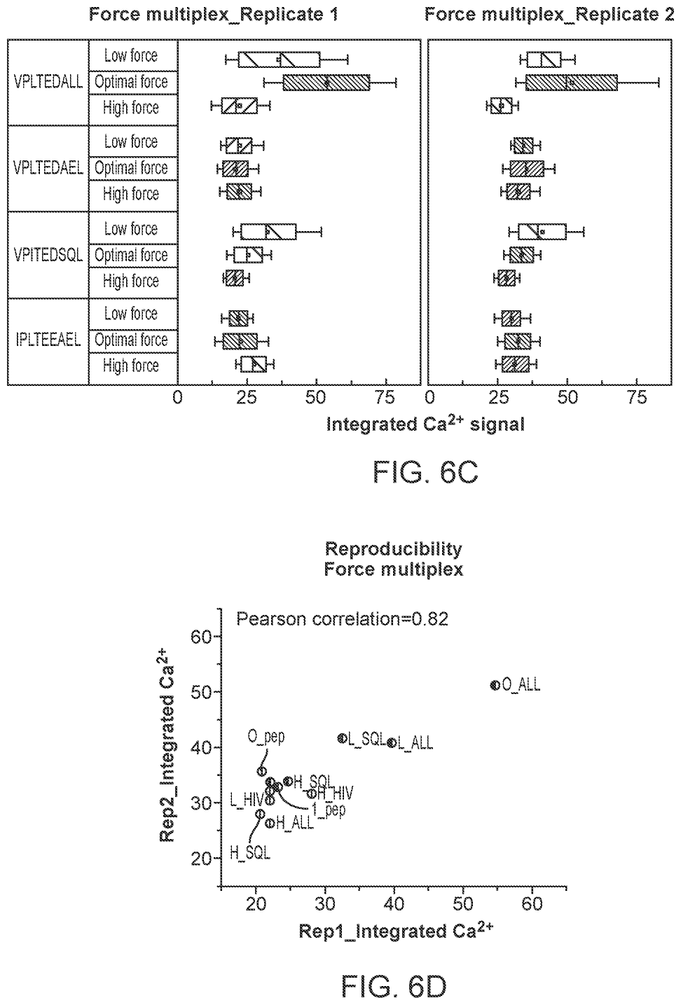

The results for force multiplexing established that TCR55 T cells bound to stimulatory (VPLTEDALL) pMHC-coated smart beads (FIG. 6A-6D) showed increased Ca²⁺ flux under low force and optimal force but not higher force. The peptide VPLTEDSQL induced increased Ca²⁺ signals when applying lower force compared to optimal force and high force. VPLTEDAEL and IPLTEEAEL could not induce any comparable Ca²⁺ signals across all three force regimes.

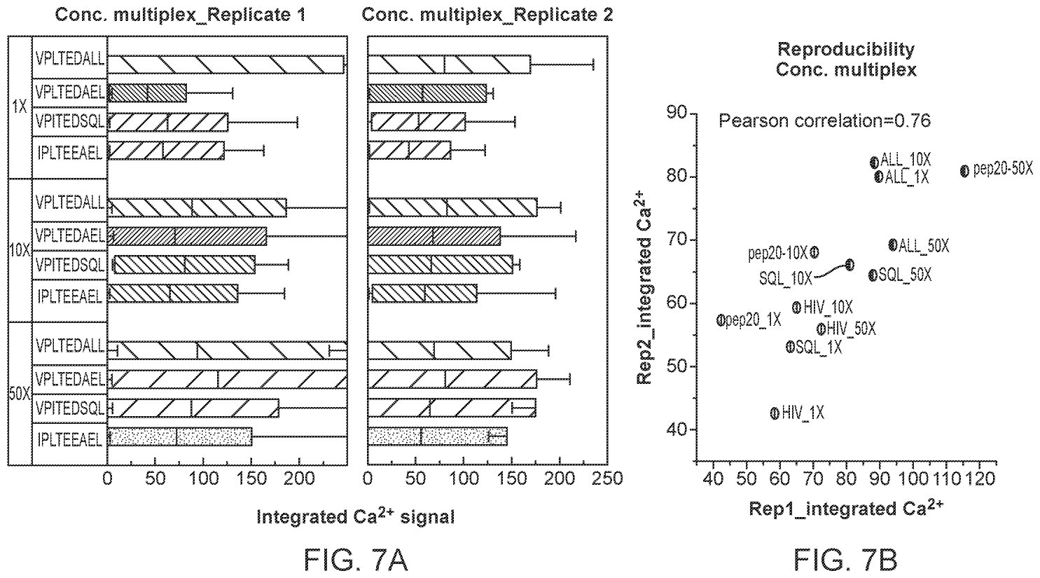

The results for concentration multiplexing established that TCR55 T cells bound to stimulatory (VPLTEDALL) pMHC-coated smart beads (FIG. 7A-7B) showed the maximum Ca²⁺ flux for 1×concentration and optimal load (˜3-4 pMHCs per cell, which is roughly 200 to 300 pMHCs per bead). However, as the concentration increased (10× and 50×), the specificity for VPLTEDALL decreased and VPLTEDAEL appeared had the most activity at 50×concentrations.

All publications, patents, and patent applications cited herein are hereby incorporated by reference with respect to the material for which they are expressly cited.

1. A method of identifying a binding moiety that binds to a target protein on the surface of a cell and mediates a force-induced signal, the method comprising:
(i) generating an arrayed configuration comprising a plurality of analysis compartments, wherein each analysis compartment in at least a portion of the arrayed configuration comprises (a) a single cell and (b) a thermo-responsive microbead comprising a thermo-responsive polymer, said microbead having a lanthanide spectral signature and, immobilized to the microbead, a binding moiety that targets a mechanosensitive molecule on the surface of the single cell;
wherein a microbead in an analysis compartment has a lanthanide spectral signature that differs from lanthanide spectral signatures of microbeads in other analysis compartments of the arrayed configuration, and the binding moiety immobilized to the microbead differs in structure or in density compared to binding moieties attached to microbeads in other analysis compartments;
(ii) incubating microbeads of (i) at a temperature of between about 25° C. and about 40° C.;
(iii) modulating the temperature to cross the phase transition temperature of the thermo-responsive polymer such that the diameter of the microbead is increased by about 1% to about 5%, thereby applying mechanical force to the cell;
(iv) measuring a signal generated in individual cells in the analysis compartments;
(v) determining the lanthanide spectral signature of a microbead contained in an analysis compartment in which a signal is detected; and
(vi) identifying the binding moiety associated with the microbead present in the analysis compartment in which a signal is measured.

2. The method of claim 1, wherein modulating the temperature comprises cooling the microbeads from a temperature above the phase-transition temperature of the thermos-responsive polymer to a temperature below the phase transition temperature, wherein the thermo-responsive polymer increases in size when cooled from the temperature above the phase-transition temperature to the temperature below the phase transition temperature.

3. The method of claim 1, wherein the thermo-responsive polymer comprises poly(N-isopropylacrylamide).

4. The method of claim 3, wherein step (ii) comprises incubating the arrayed configuration at a temperature in a range from about 37° C. to about 40° C.

5. The method of claim 4, wherein step (iii) comprises decreasing the temperature to a temperature of about 32° C. to about 34° C.

6. The method of claim 3, wherein step (ii) comprises incubating the arrayed configuration at about 37° C. and step (iii) comprises decreasing the temperature to about 34° C.

7. The method of claim 1, wherein modulating the temperature comprises heating the microbeads from a temperature below the phase-transition temperature of the thermos-responsive polymer to a temperature above the phase transition temperature, wherein the thermos-responsive polymer increases in size when heated from the temperature below the phase-transition temperature to the temperature above the phase-transition temperature.

8. The method of claim 1, wherein the arrayed configuration comprises compartments in which the microbeads have different phase transition temperatures.

9. The method of claim 1, wherein the mechanosensitive molecule comprises a T-cell receptor.

10. The method of claim 9, wherein the binding moiety immobilized on the thermo-responsive microbead is a peptide-loaded MHC complex and the single cell in each compartment is a T cell.

11. The method of claim 10, wherein the T-cell is a CD8⁺ T cell or CD4⁺ T cell.

12. (canceled)

13. The method of claim 10, wherein the peptide loaded onto the MHC complex is a candidate T-cell epitope from a cancer antigen.

14. The method of claim 10, wherein step (iv) comprises measuring calcium flux.

15. The method of claim 1, wherein the binding moiety is immobilized to the microbead via interaction of biotin and streptavidin.

16. A thermo-responsive microbead having a lanthanide spectral signature and a binding moiety immobilized to the microbead, wherein the binding moiety targets a mechanosensitive molecule on the surface of a cell and the microbead comprises a thermo-responsive polymer having a phase transition temperature in the range of about 25° C. to about 45° C.: optionally wherein the binding moiety is immobilized to the microbead via interaction of biotin and streptavidin.

17. The thermo-responsive microbead of claim 16, wherein the thermo-responsive polymer comprises poly(N-isopropylacrylamide).

18. The thermo-responsive microbead of claim 16, wherein the binding moiety targets a T-cell receptor.

19. The thermo-responsive microbead of claim 18, wherein the binding moiety immobilized on the thermo-responsive microbead is a peptide-loaded MHC complex, wherein the peptide comprises a candidate T cell epitope, optionally wherein the candidate T-cell epitope is a CD8⁺ T-cell epitope or CD4⁺ T-cell epitope; and/or is from a cancer antigen.

20-23. (canceled)

24. An arrayed configuration comprising a plurality of analysis compartments, wherein each a portion of the analysis compartments comprises (a) a single cell and (b) a thermo-responsive microbead of claim 16, wherein the lanthanide spectral signature of a microbead differs from lanthanide spectral signatures of microbeads in the other analysis compartments, and the binding moiety immobilized to the microbead differs in structure or density from the binding moieties immobilized to microbeads in other compartments; and/or the arrayed configuration comprises analysis compartments in which microbeads present in different compartments differ in phase transition temperature.

25. A method of identifying an MHC epitope that activates a T-cell, the method comprising:
i) generating an arrayed configuration comprising a plurality of analysis compartments, wherein at least a portion of the arrayed configuration comprises (a) a single T-cell and (b) a thermo-responsive microbead comprising a thermo-responsive polymer and a peptide-loaded MHC complex immobilized on the microbead;
wherein a microbead in an analysis compartment has a lanthanide spectral signature that differs from lanthanide spectral signatures of microbeads in other analysis compartments, and the peptide-loaded MHC complex comprises a peptide that differs in sequence and/or the peptide-loaded MHC complex is present on the microbead at a different density compared to the peptide-loaded MHC complexes immobilized to microbeads in other analysis compartments;
(ii) incubating microbeads of (i) at a temperature of between about 25° C. and about 40° C.;
(iii) modulating the temperature to cross the phase transition temperature of the thermos-responsive polymer such that the diameter of the thermos-responsive microbead is increased by about 1% to about 5%, thereby applying shear force to the cell;
(iv) measuring a signal generated by individual T-cells in the analysis compartments;
(v) determining the lanthanide spectral signature of a microbead contained in an analysis compartment in which a signal is detected; and
(vi) identifying the sequence of the peptide of the peptide-loaded MHC complex and/or the density of the peptide-loaded MHC complex associated with the microbead in an analysis compartment in which a signal is detected, thereby identifying a T-cell epitope that activates a T cell.

26. The method of claim 25, wherein the thermo-responsive polymer comprises poly(N-isopropylacrylamide), optionally wherein step (ii) comprises incubating the microbeads at a temperature of about 37° C. to about 40° C.; or step (iii) comprises decreasing the temperature to about 32° C. to about 34° C.: or step (i) comprises incubating the microbeads at a temperature of about 37° C. and step (ii) comprises decreasing the temperature from about 37° C. to about 34° C.

27-29. (canceled)

30. The method of claim 25, wherein step (iv) comprises measuring calcium flux; and/or the T cell is a CD8⁺ T cell or a CD4⁺ T cell.

31-32. (canceled)

33. The method of claim 25, wherein the arrayed configuration comprises at least a first compartment that contains a peptide-loaded MHC complex in which the peptide sequence is the same as the sequence of a peptide-loaded MHC complex present in a second compartment and the density of the peptide-loaded MHC complexes immobilized to the microbead in the first compartment is different from the density of the peptide-loaded MHC complexes immobilized to the microbead in the second compartment.

34. The method of claim 25, wherein the method the arrayed configuration comprises at least a first compartment that contains a peptide-loaded MHC complex in which the peptide sequence and the density of the peptide-loaded MHC complex immobilized to the microbead is the same as the peptide sequence and the density of the peptide-loaded MHC complex in a second compartment, and the microbead in the first compartment differs in its phase transition temperature compared to the microbead in the second compartment.
